SEQUENCE LISTING (10 sequences)

SEQ ID NO: 1 - protein, 20 aa, synthetic construct
GACCCAGTCCCTACTTATTG

SEQ ID NO: 2 - protein, 20 aa, synthetic construct
GAGAGACATATTCAAAAGTG

SEQ ID NO: 3 - protein, 20 aa, synthetic construct
CCATTTTTGGACCACAAAAA

SEQ ID NO: 4 - protein, 20 aa, synthetic construct
CCACTAGTCTCTAGTCAGTG

SEQ ID NO: 5 - protein, 20 aa, synthetic construct
GGTAATAAACACCACGTGTG

SEQ ID NO: 6 - protein, 20 aa, synthetic construct
AGAACTCAATCATACACTAA

SEQ ID NO: 7 - protein, 20 aa, synthetic construct
GACCCAGTCCCTACTTATTG

SEQ ID NO: 8 - protein, 20 aa, synthetic construct
GAGAGACATATTCAAAAGTG

SEQ ID NO: 9 - protein, 20 aa, synthetic construct
ATGGAAAGTGGAGTTTATTC

SEQ ID NO: 10 - protein, 20 aa, synthetic construct
GAAAGTGAGTTCAGAGTTTA

(19) United States
(12) Patent Application Publication
(10) Pub. No.: US 2025/0297331 A1
(43) Pub. Date: Sep. 25, 2025

(54) METHODS FOR DETECTING GENOMIC VARIANTS OF SARS-COV-2 IN MULTIPLEX ASSAYS

(71) Applicants: Björn ROTTER, Frankfurt am Main (DE); GENXPRO GMBH, Frankfurt am Main (DE)

(72) Inventors: Björn ROTTER, Frankfurt am Main (DE); Klaus HOFFMEIER, Frankfurt am Main (DE)

(73) Assignee: GENXPRO GMBH, Frankfurt am Main (DE)

(21) Appl. No.: 18/845,655

(22) PCT Filed: Mar. 10, 2023
(86) PCT No.: PCT/EP2023/056232

§ 371 Date: Sep. 10, 2024

Related U.S. Application Data
(60) Provisional application No. 63/318,645, filed on Mar. 10, 2022.

(51) Int. Cl. C12Q 1/70; C12Q 1/6825; C12Q 1/686

(52) U.S. Cl. CPC C12Q 1/701; C12Q 1/6825; C12Q 1/686; C12Q 2600/16

(57) ABSTRACT

Methods are provided for detecting the presence of a specific genomic variant in a multiplex assay and distinguishing it from other genomic variants by interrogating one or more genomic loci specific to a particular genomic variant, and one or more semi-specific genomic loci present in in at least two genomic variants. The methods are useful for detecting a SARS-CoV-2 variant in a sample.

### CROSS-REFERENCE TO RELATED APPLICATIONS

The present application claims priority to U.S. Provisional Patent Application 63/318,645, filed Mar. 10, 2022, the contents of which are incorporated by reference herein in its entirety.

### SEQUENCE LISTING

The application contains a Sequence Listing which has been submitted electronically in ST.26 Sequence listing XML format and is hereby incorporated by reference in its entirety. Said ST.26 Sequence listing XML, created on Feb. 28, 2023, is named ROTTER-001.xml and is 9,602 bytes in size.

### TECHNICAL FIELD

The present disclosure relates to methods for specifically and semi-specifically detecting genomic variants in multiplex assays.

### BACKGROUND

Detection of nucleic acid sequence variants of viruses, bacteria, fungi, and mammalian genes informs treatment decisions and is important for tracking the transmission of viral, bacterial or fungal infections in a population, as well as for development of vaccines and biopharmaceuticals.

Multiplex polymerase chain reaction (PCR) is used to amplify and identify more than one target nucleic acid sequence (such as DNA or RNA) in a sample. Multiplex Quantitative Real Time PCR (qPCR) is used to monitor amplification of target nucleic acid sequences during a polymerase chain reaction. The amplified nucleic acid sequences or amplicons are identified using multiple signals, such as for example, fluorophores in a one-to-one ratio of fluorophore per target nucleic acid sequence. However, use of multiple fluorophores is not desirable due to cost and the limitations of multiplex PCR machine capabilities. Typical multiplex PCR machines have up to four channels which cannot accommodate more than 4 fluorophores. Using a one-to-one ratio of fluorophore per target nucleic acid with a four channel PCR machine limits identification of target nucleic acids sequences in a sample to 3 target sequences and one control sequence.

Viruses, bacteria and fungi evolve due to genetic mutations that occur during replication of the viral, bacterial or fungal genome leading to variants. More efficient and less costly methods for detecting multiple nucleic acids in multiplex systems are needed, particularly, for detecting viral, bacterial and fungal variants. For example, SARS-CoV-2 is a coronavirus which causes the infectious disease COVID-19. Due to the COVID-19 pandemic, and the likelihood that SARS-CoV-2 variants will become endemic, there is a need for new and improved methods to efficiently detect SARS-CoV-2 viral variants.

### BRIEF SUMMARY

Provided herein are surprisingly efficient methods for detecting a polynucleotide variant in a sample using a combination of signals to detect loci that are specific and/or semi-specific for that polynucleotide variant. In some embodiments, the signals are fluorophores wherein one fluorophore signal is specific to a particular variant and one or more semi-specific fluorophore signals are used to detect variants that have a locus in common.

The methods provided herein advantageously utilize limited resources to detect genomic variants in a multiplex assay. The methods of the present invention eliminate the need for all probes to be labeled with a unique signal, and also eliminate the need to label a probe with more than one signal. In one embodiment, methods are provided for detecting multiple genomic variants with a lesser number of fluorophores. In some aspects, the methods of the invention can be used to detect three genomic variants with only two fluorophores and using only two channels of a PCR machine.

In some embodiments, the multiplex assay is a PCR assay.

In other embodiments, the multiplex assay is a lateral flow assay. In another aspect, a lateral flow dipstick (LFP) assay can be used to detect genomic variants in accordance with the methods herein.

In one embodiment, methods are provided for detecting viral variants in a sample.

In one aspect, methods are provided for detecting viral variants in a sample including, but not limited to, coronaviruses, human immunodeficiency viruses, hepatoviruses, human papillomaviruses, and herpes simplex viruses.

In another aspect, methods are provided for detecting SARS-Cov-2, SARS-COV, or MERS-CoV variants.

In another aspect, the methods provided herein can be used to detect two or more viral variants in a sample, as well as variants having a combination of mutations present in two other variants. For example, the methods of the invention can be used to determine if a patient is infected with a SARS-CoV-2 variant having a combination of mutations from an Omicron variant and a Delta variant, coinfected with two SARS-CoV-2 variants, or coinfected with an influenza virus and SARS-CoV-2.

In one aspect, methods and kits are provided for detecting a SARS-CoV-2 variant, such as Omicron BA.1, Omicron BA1.1, Omicron BA.2 or Delta, in a sample.

In another embodiment, methods are provided for detecting bacterial variants in a sample.

In another embodiment, the methods herein can be used to detect human leukocyte antigen (HLA) variants useful for matching organ, bone marrow, or cord blood donors with recipients.

In other embodiments, methods for treating a patient infected with SARS-CoV-2 are provided comprising performing a multiplex assay such as a PCR assay or lateral flow assay to detect a SARS-CoV-2 variant in a patient comprising amplifying a SARS-CoV-2 polynucleotide having one or more loci, and detecting two or more of the loci with one signal, wherein the detection of one or more signals identifies the presence of a particular SARS-CoV-2 variant in the patient sample, and treating the patient with a therapy effective for treating that particular SARS-CoV-2 variant. In one aspect, the therapy for treating the SARS-CoV-2 variant comprises a biologic such as an antibody or monoclonal antibody therapy. In another aspect, the therapy for treating the SARS-CoV-2 variant comprises a small molecule. In still another aspect, a therapy for treating a SARS-CoV-2 variant comprises combinations of monoclonal antibodies, combinations of small molecules, or combinations of monoclonal antibodies and small molecules.

The present invention may be embodied in other specific forms without departing from the spirit or essential attributes thereof. Thus, the above embodiments should not be considered limiting. Any and all embodiments of the present invention may be taken in conjunction with any other embodiment or embodiments to describe additional embodiments. Each individual element of the embodiments is its own independent embodiment. Furthermore, any element of an embodiment is meant to be combined with any and all other elements from any embodiment to describe an additional embodiment. In addition, the present invention encompasses combinations of different embodiment, parts of embodiments, definitions, descriptions, and examples of the invention noted herein.

### BRIEF DESCRIPTION OF THE DRAWINGS

FIG. 1 is a schematic of an exemplary method of the present invention for detecting a genomic variant. The schematic shows a first genomic variant 101 wherein specific Locus 104 is detected by a Signal X-labeled probe; a second genomic variant 102 wherein specific Locus 105 is also detected by a Signal X-labeled probe and semi-specific Locus 106 is detected by a Signal Y-labeled probe; and a third genomic variant 103 wherein semi-specific Locus 106 is detected by a Signal Y-labeled probe and specific Locus 107 is also detected by a Signal Y-labeled probe.

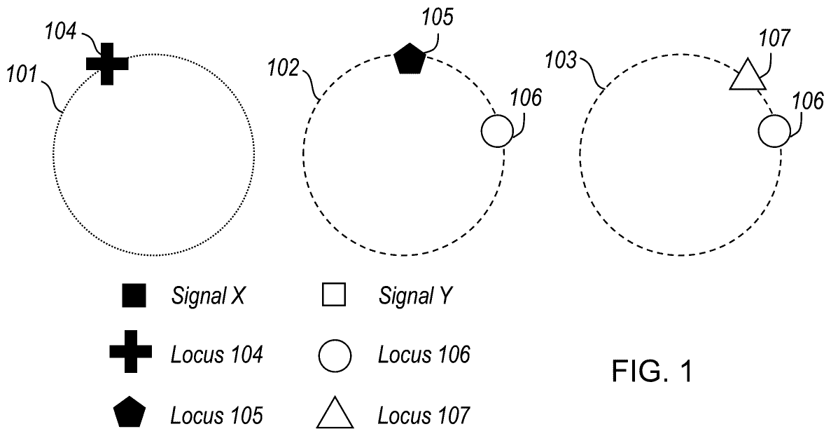

FIG. 2 is a schematic of a method of the present invention for detecting the SARS-CoV-2 genomic variants Omicron BA.1, Delta, and Omicron BA.2. The schematic shows Omicron BA.1 201 wherein specific locus D143 is detected by a fluorophore X-labeled probe; the Delta variant 202 wherein specific locus 205 (D157) is also detected by a fluorophore X-labeled probe and semi-specific locus 206 (wt143) is detected by a fluorophore Y-labeled probe; and the Omicron BA.2 variant 203 wherein semi-specific locus 206 (wt143) is detected by a fluorophore Y-labeled probe and specific locus 207 (D24) is also detected by a fluorophore Y-labeled probe.

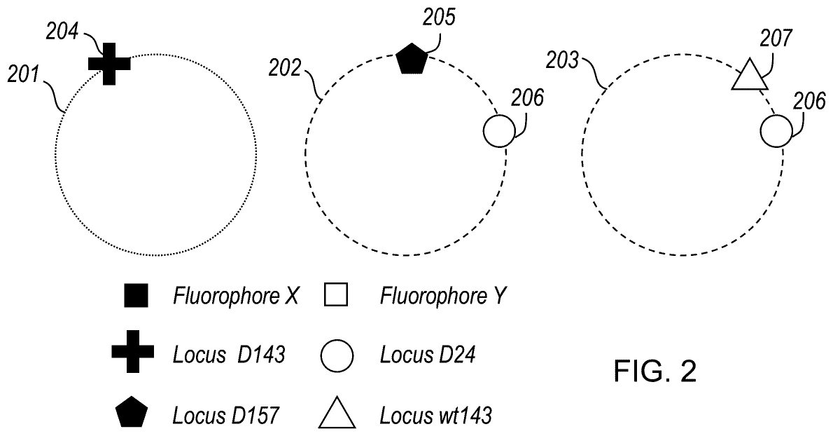

FIG. 3 shows representative possible combinations of signals for detecting seven genomic variants using six loci and three fluorophores (X, Y and Z). In this embodiment, each representative combination has at least one fluorophore signal specific to a particular variant and four or more semi-specific fluorophore signals when variants have a locus in common. In another embodiment, one of the possible combinations is a lack of signal at all six loci indicated in FIG. 3 as an eighth variant, Variant H, which indicates that a genomic variant is present in a sample, but that variant does not include any of the six loci used in the assay.

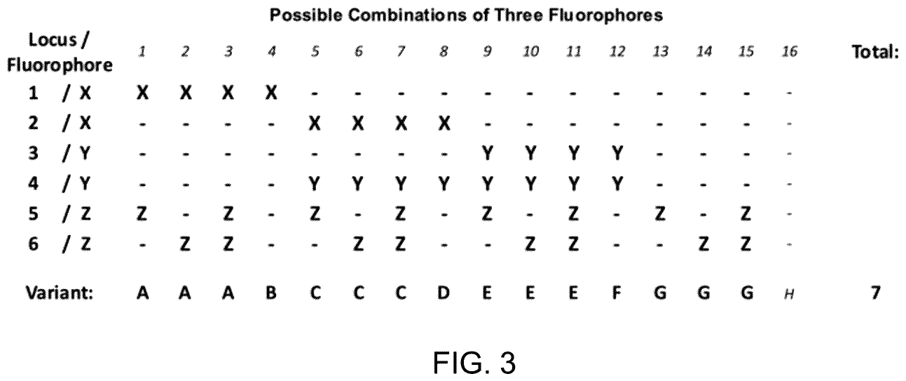

FIG. 4 shows representative combinations of signals for detecting three genomic variants (1, 2, and 3) using four loci and two signals (X and Y). Each representative combination has at least one signal specific to a particular variant and one or more semi-specific signals when two variants have a locus in common.

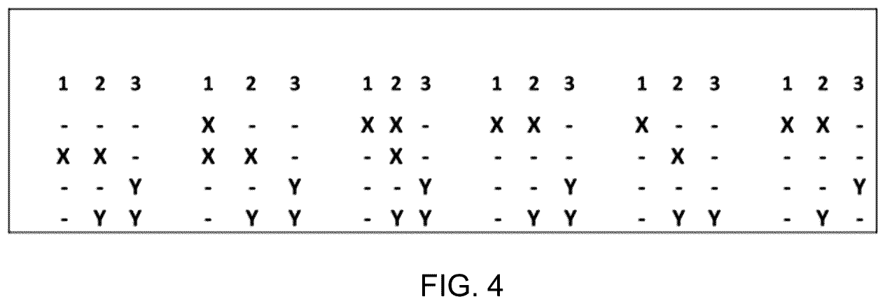

FIG. 5 shows a comparison of LiPA reaction patterns of 16 separate HLA-C loci for diploid DNA identified as Cw05/08 (top), haploid DNA after haplotype-specific extraction (center) and haploid DNA identified as the Cw*0504 allele after haplotype-specific extraction, and haploid DNA identified as the Cw*0504 allele after haplotype-specific extraction and whole genome amplification (bottom).

FIG. 6 is a graph showing the change in fluorescence delta (Δ) Rn versus the cycle number during PCR amplification of a sample containing SARS-CoV-2 Omicron BA.1 variant.

FIG. 7 is a graph showing the change in fluorescence delta (Δ) Rn versus the cycle number during PCR amplification of a sample containing SARS-CoV-2 Omicron BA.2 variant.

FIG. 8 is a graph showing the change in fluorescence delta (Δ) Rn versus the cycle number during PCR amplification of a sample containing SARS-CoV-2 Delta variant.

### DETAILED DESCRIPTION

Provided herein are methods for efficiently identifying the presence of a polynucleotide variant in a sample using a combination of signals to detect loci that are specific and/or semi-specific for that polynucleotide variant. In some embodiments, the signals are fluorophores.

The methods of the present invention eliminate the need for all probes to be labeled with a unique signal. The methods herein also eliminate the need to label a probe with more than one signal.

In one embodiment, methods are provided for detecting three genomic variants with only two fluorophores and using only two channels of a PCR machine.

In some aspects, four different genomic or polynucleotide loci are examined per PCR assay wherein the four different loci can be detected with only two fluorophores.

In some embodiments of the present invention, the presence of a genomic variant is detected by interrogating four loci wherein one signal is used to detect a first locus and second locus, and a second signal is used to detect a third locus and fourth locus wherein the fourth locus is a semi-specific locus that is shared by two of the genomic variants. In the methods provided herein, for each genomic variant to be detected, at least one uniquely identifying locus is examined.

In another aspect, provided herein are genomic variant detection methods in which an additional signal is utilized to detect an additional polynucleotide sequence, for example, a control locus that is present in all variants being tested, or a host gene to confirm the sample was extracted from that host, such as a human gene to confirm the sample tested was a human sample.

Examples of fluorophores suitable for use with the methods of the present invention are, but not limited to, 5(6)-carboxyfluorescein (FAM), Hexachloro-Fluorescein (HEX), carboxy-X-rhodamine (ROX), cyanine5 (CY5), and NED™, VIC®, PET™ and LIZ® dyes from Applied Biosystems.

In some aspects, the probes include a quencher. A fluorescent signal is generated when the fluorophore is separated from the quencher during PCR extension and amplification of the polynucleotide of interest. For example, during PCR, the exonuclease activity of Taq polymerase cleaves off the fluorophore separating it from the quencher. Examples of suitable quenchers for use in the methods provided herein are Black Hole quencher (BHQ), BMN-Q535, BMN-Q620, Dabcyl, TAMRA (tetramethylrhodamine), Deep Dark quencher (DDQ).

In one aspect, the methods for detecting genomic variants can be used to detect viral variants, including but not limited to Coronavirus variants such as, for example, SARS-CoV-2, SARS-COV, and MERS-COV. Examples of other viral variants that can be detected using the methods provided herein are HIV, hepatitis, human papilloma virus, and herpes simplex virus.

Provided herein are SARS-CoV-2 Delta/Omicron variant detection assays. In one aspect, the assay is a reagent system, based on quantitative reverse transcriptase (qRT) PCR technology, for the detection of RNA specific to SARS-CoV-2 and to SARS-CoV-2 Variants of Concern (VoC) that cause the Coronavirus Disease 2019 (COVID-19).

Quantitative polymerase chain reaction (qPCR) technology utilizes an enzyme (reverse-transcriptase, RT) to convert RNA to complementary DNA (DNA), from which specific target sequences are then amplified and targeted with specific probes for the detection of their copy number in the specimen.

In one aspect, SARS-CoV-2 Delta/Omicron Variant detection assays provided herein detect SARS-CoV-2 and simultaneously discriminate between the Delta variant, and the BA.1 and BA.2 variants of Omicron. In another embodiment, other SARS-CoV-2 variants and subvariants, for example, Omicron BA1.1, can be detected using the methods and kits provided herein.

SARS-CoV-2 is a single-stranded RNA-enveloped virus. The SARS-CoV-2 genome is 29,991 bp (GenBank No. MN908947) and encodes 9860 amino acids. See, Zhu et al. N. Engl. J. Med. 2020, 382:727-733. The SARS-CoV-2 spike protein(S) on the virus envelope includes subunits S1 and S2. Subunit S1 includes a receptor binding domain that recognizes and binds to the main host cellular receptor angiotensin-converting enzyme 2 (ACE2). The S2 subunit mediates fusion of the viral envelope with a host cell. Nucleic acid sequences of SARS-CoV-2 variants are available on the Nextstrain project website at Nextstrain.org and the Covariants.org website, Emma B. Hodcroft. 2021 “CoVariants: SARS-CoV-2 Mutations and Variants of Interest” and other publicly available databases.

The SARS-CoV-2 host cell receptor ACE2 is widely expressed in vertebrates. In the methods for detecting a SARS-CoV-2 variant in a sample provided herein, a sample or specimen can be from a vertebrate including, but not limited to, a human or other mammal such as, but not limited to, dogs, cats, bats, civets, pangolins, livestock, rodents and deer. In another aspect, the sample to be detected using the methods of the present invention can be extracted from a bird or reptile.

Patient samples to be tested in the methods provided herein can be from a human or other mammal. In one aspect, in the methods provided herein for treating a patient, the patient is a human patient. In another aspect, the patient can be a veterinary patient.

Methods provided herein determine the strain or variant of SARS-CoV-2 by detecting variant specific nucleic acid deletions. Examples of SARS-CoV-2 loci particularly useful for performing the methods provided herein are SARS-CoV-2 spike protein loci including, but not limited to, deletion of valine at position 143 in the SARS-Cov-2 spike protein (D143), deletion of leucine at position 24 in the SARS-Cov-2 spike protein (D24), deletion of phenylalanine at position 157 in the SARS-Cov-2 spike protein (D157), and valine at position 143 of the wild type spike protein (WT143).

In other embodiments four different polynucleotide loci are interrogated per multiplex PCR assay wherein a first polynucleotide locus of a first genomic variant is detected by a probe labeled with a first signal; a second polynucleotide locus of a second genomic variant is detected by a probe labeled with the first signal; a third polynucleotide locus of a third genomic variant is detected by a probe labeled with a second signal; and a fourth polynucleotide locus present in both the second and third genomic variants is detected by a probe labeled with the second signal to determine which genomic variant is present in a sample.

Primers suitable for use in the methods of the present invention are single-stranded oligonucleotides, capable of acting as a point of initiation of synthesis along a complementary nucleic acid strand to produce a primer extension product (amplicon) to amplify a nucleic acid sequence of interest. In some embodiments, a method may be provided for detecting a SARS-CoV-2 variant in a sample (such as an animal sample, such as a human sample, etc.) The method may include adding three primer sets to a multiplex PCR machine wherein each primer set is capable of extending and duplicating a polynucleotide sequence comprising a SARS-Cov-2 variant locus. The method may include adding a plurality of probes to the multiplex PCR machine. The method may include adding a first probe to the multiplex PCR machine wherein the first probe is labeled with a first signal capable of binding to a first SARS-Cov-2 locus. The method may include adding a second probe to the multiplex PCR machine wherein the second probe is labeled with a second signal capable of binding to a second SARS-Cov-2 locus but is not capable of binding to the first SARS-Cov-2 locus. The method may include adding a third probe to the multiplex PCR machine wherein the third probe is labeled with the first signal wherein the third probe is capable of binding to a third SARS-Cov-2 locus but is not capable of binding to the first or second SARS-Cov-2 loci. The method may include adding a fourth probe to the multiplex PCR machine wherein the fourth probe is labeled with the second signal wherein the fourth probe is capable of binding to a fourth SARS-Cov-2 locus present in both of the second and third SARS-Cov-2 variants but not present in the first SARS-Cov-2 variant. The method may include performing PCR in the multiplex PCR machine. The method may include detecting the presence of only the first signal, only the second signal, or both the first and second signals. Detection of only the first signal identifies the presence of the first SARS-Cov-2 variant in the sample. Detection of only the second signal identifies the presence of the second SARS-Cov-2 variant in the sample. Detection of both the first and second signals identifies the presence of the third SARS-Cov-2 variant in the sample.

In some embodiments, the first and second signals may be fluorophores. In some embodiments, the first and second fluorophores may be FAM, HEX, ROX, CY5, NED, VIC, PET, or LIZ. For example, in some embodiments, the first signal may be ROX and the second signal may be CY5.

In some embodiments, the SARS-Cov-2 locus may be selected from a locus within the group consisting of an Omicron variant, a Delta variant, and combinations thereof. In some embodiments, the SARS-Cov-2 variant may be selected from the group consisting of Omicron BA.1, Omicron BA1.1, Omicron BA.2 and Delta. In some embodiments, the first SARS-Cov-2 locus may be the D143 locus in the Omicron BA.1 variant. In some embodiments, the second SARS-Cov-2 locus may be D24 in the Omicron BA.2 variant. In some embodiments, the third SARS-Cov-2 locus may be D157 in the Delta variant. In some embodiments, the fourth SARS-Cov-2 locus may be WT143 present in the Omicron BA.2 variant and the Delta variant.

In some embodiments, a method for treating a patient for SARS-Cov-2 may be provided. The method may include obtaining a sample from the patient. The method may include adding the sample and primers to a multiplex PCR machine wherein each primer is capable of extending and duplicating a SARS-Cov-2 variant locus. The method may include adding a plurality of probes to the multiplex PCR machine. A first probe may be labeled with a first signal capable of binding to a first SARS-Cov-2 locus. A second probe may be labeled with a second signal capable of binding to a second SARS-Cov-2 locus but is not capable of binding to the first SARS-Cov-2 locus. A third probe may be labeled with the first signal wherein the third probe is capable of binding to a third SARS-Cov-2 locus but is not capable of binding to the first or second SARS-Cov-2 loci. A fourth probe may be labeled with the second signal wherein the fourth probe is capable of binding to a fourth SARS-Cov-2 locus present in both of the second and third SARS-Cov-2 variants but not present in the first SARS-Cov-2 variant. The method may include performing PCR in the multiplex PCR machine. The method may include detecting the presence of only the first signal, only the second signal, or both the first and second signals.

Detection of only the first signal identifies the presence of the first SARS-Cov-2 variant (e.g., Omicron BA.1) in the sample. If the first SARS-CoV-2 variant is detected, the method may include treating the patient with a therapy effective against the first SARS-CoV-2 variant. Detection of only the second signal identifies the presence of the second SARS-Cov-2 variant (e.g., Omicron BA.2) in the sample. If the second SARS-CoV-2 variant is detected, the method may include treating the patient with a therapy effective against the second SARS-CoV-2 variant. Detection of both the first and second signals identifies the presence of the third SARS-Cov-2 variant (e.g., Delta) in the sample. If the third SARS-CoV-2 variant is detected, the method may include treating the patient with a therapy effective against the third SARS-CoV-2 variant. In some embodiments, the therapy effective against the first SARS-CoV-2 variant, second SARS-CoV-2 variant, and/or third SARS-CoV-2 variant is a monoclonal antibody, a small molecule, or a combination thereof.

In one embodiment of the invention, a mixture of primers and probes are used in methods and kits for detecting a SARS-CoV-2 variant in a sample wherein (a) the forward and reverse primers for amplifying the Omicron BA.1 variant comprise a nucleic acid sequence of SEQ ID NO: 1 (GACCCAGTCCCTACTTATTG) and a nucleic acid sequence of SEQ ID NO: 2 (GAGAGACATATTCAAAAGTG), respectively, (b) the labeled probe for detecting the Omicron BA.1 variant (D143 locus) is a nucleic acid sequence of SEQ ID NO: 3 (CCATTTTTGGACCACAAAAA), (c) the forward and reverse primers for amplifying Omicron BA.2 are a nucleic acid sequence comprising SEQ ID NO: 4 (CCACTAGTCTCTAGTCAGTG) and SEQ ID NO: 5 (GGTAATAAACACCACGTGTG), respectively, (d) the labeled probe for detecting the Omicron BA.2 variant (D24 locus) is a nucleic acid sequence of SEQ ID NO: 6 (AGAACTCAATCATACACTAA), (e) the forward and reverse primers for detecting the Delta variant are a nucleic acid sequence of SEQ ID NO: 7 (GACCCAGTCCCTACTTATTG) and a nucleic acid sequence of SEQ ID NO: 8 (GAGAGACATATTCAAAAGTG), respectively, (f) the labeled probe for detecting the Delta variant (D157 locus) is a nucleic acid sequence of SEQ ID NO: 9 (ATGGAAAGTGGAGTTTATTC), and the labeled drop-off probe for the BA.1 variant (WT143 locus) which provides a positive signal when Omicron BA.2 or Delta are in a sample but a negative signal when BA.1 variant is in the sample is a nucleic acid sequence of SEQ ID NO:10 (GAAAGTGAGTTCAGAGTTTA).

In another embodiment, the methods herein can be used to detect human leukocyte antigen (HLA) variants useful for matching organ, bone marrow or cord blood donors with recipients.

Quantitative RT-PCR can be used in performing the methods of the present invention. See for example, Toptan et al., Optimized qRT-PCR Approach for the Detection of Intra- and Extra-Cellular SARS-CoV-2 RNAs, Int J Mol Sci. 2020 Jun. 20; 21 (12): 4396. See also Sambrook, Fritsch & Maniatis, Molecular Cloning; A Laboratory Manual, Second Edition, (1989) (hereinafter “Maniatis”); Oligonucleotide Synthesis (M. J. Gait, ed., 1984); Nucleic Acid Hybridization (B. D. Hames & S. J. Higgins, eds., 1984); A Practical Guide to Molecular Cloning (B. Perbal, 1984); and a series, Methods in Enzymology (Academic Press, Inc.). Multiplex PCR methods are described and incorporated by reference in Abbs, S and Bobrow, M, “Analysis of quantitative PCR for the diagnosis of deletion and duplication carriers in the dystrophin gene” Journal of Medical Genetics (1992) 29 (3): 191-96; Morlan, J., et al. “Mutation Detection by Real-Time PCR: A Simple, Robust and Highly Selective Method” PLOS ONE (2009) 4 (2) e4584; and Elnifro, et al., Clin Microbiol Rev. (2000) October; 13 (4): 559-570.

Examples of suitable PCR systems for use in performing the methods herein are APPLIED BIOSYSTEMS 7500 Real-Time PCR System (THERMO FISHER®, Foster City, California) STEPONE™ and STEPONEPLUS™ Real-Time PCR Systems (THERMO FISHER®, Foster City, California) ROTOR-GENE® (QUIAGEN®, Germantown, MD), Mic Real-Time PCR magnetic induction cycler (Bio Molecular Systems, Upper Coomera, Australia), CFX Real-Time PCR systems (BIO-RAD, Hercules, California) (LightCycler® and Light Cycler 480® Real-Time PCR Systems (ROCHE, Rotkreuz, Switzerland).

In one aspect, one or more control signals are included in a multiplex assay. Examples of control signals are probes that confirm a sample is a human specimen such as RNase P (RP), and/or probes that confirm the presence of a sequence common to all nucleic acids being tested in a multiplex assay such as the SARS-CoV-2 Nucleocapsid (NP), or the Nucleocapsid (NP) N1 fragment for universal SARS-CoV-2 detection.

The methods provided herein are surprisingly efficient in detecting the presence of a specific genomic variant and distinguishing it from other genomes by interrogating one or more genomic loci specific to a particular genomic variant and one or more semi-specific genomic loci present in in at least two or more of the genomic variants.

Using the methods provided herein, PCR machines having two to four fluorophore channels are used to test for the presence of multiple genomic variants in a single multiplex assay that would otherwise not be possible, therefore saving cost, time and reagents as well as reducing errors, and simplifying workflow.

In one aspect, several different genomic loci are examined per assay.

In another aspect, at least one genomic locus is examined that produces the same signal in at least two of the genomic variants (semi-specific signal).

In still another aspect, genomic loci are examined that generate a signal in only one genomic locus (specific signal).

In one embodiment, one genomic variant is identified by a single specific signal; and another genomic variant is identified by the combination of specific and semi-specific signals.

In one embodiment three different genomic variants of SARS-CoV-2 (Omicron BA.1, Omicron BA.2, Delta) are recognized through the use of only two signals. The two signals can be two fluorophores such as ROX and CY5. Table 1 below shows the signals for detection of the Omicron BA.1 and BA.2, and Delta variants using two fluorophores, ROX and CY5 in a method of the present invention. In this embodiment, a positive ROX signal and negative CY5 signal indicates the Omicron BA.1 variant is present in the sample. A positive CY5 signal and negative ROX signal indicates the Omicron BA.2 is present in the sample, and generation of both ROX and CY5 signals indicates the Delta variant is present in the sample.

TABLE 1
Detection of SARS-CoV-2 genomic variants

| | |
|---|---|
| Locus/ | SARS-CoV-2 Genomic Variant |

| Probe Label | BA.1 | BA.2 | Delta |
|---|---|---|---|
| D143/ROX | ROX | No | No |
| D157/ROX | No | No | ROX |
| D24 /CY5 | No | CY5 | No |
| WT143/CY5 | No | CY5 | CY5 |

D143, D157, D24, and WT143 represent genetic loci that are used to identify SARS-CoV-2 genomic variants.

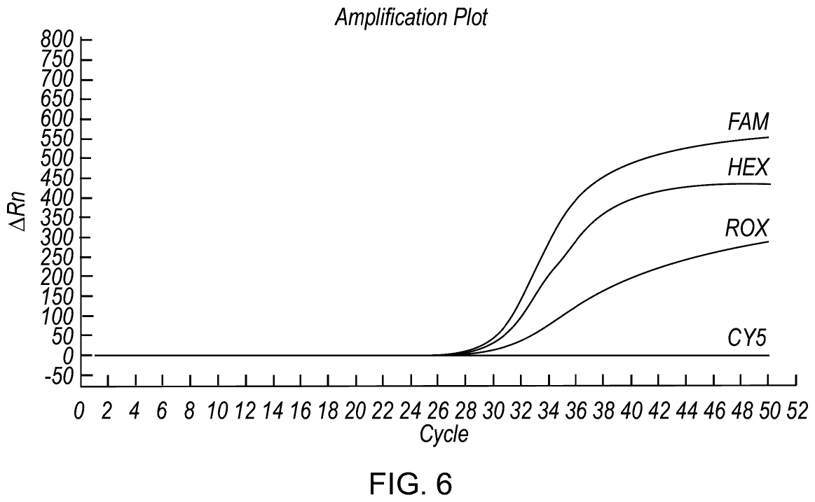

FIG. 6 is a graph showing the change in fluorescence ΔRn versus the cycle number during PCR amplification of a sample containing SARS-CoV-2 Omicron BA.1 variant. The graph shows ROX fluorescent signal indicating Omicron BA.1 variant is present in the sample. The FAM signal detected RNase P (RP) confirming the sample is a human specimen, and the HEX signal detected Nucleocapsid (NP) in the sample confirming the presence of SARS-CoV-2 in the sample.

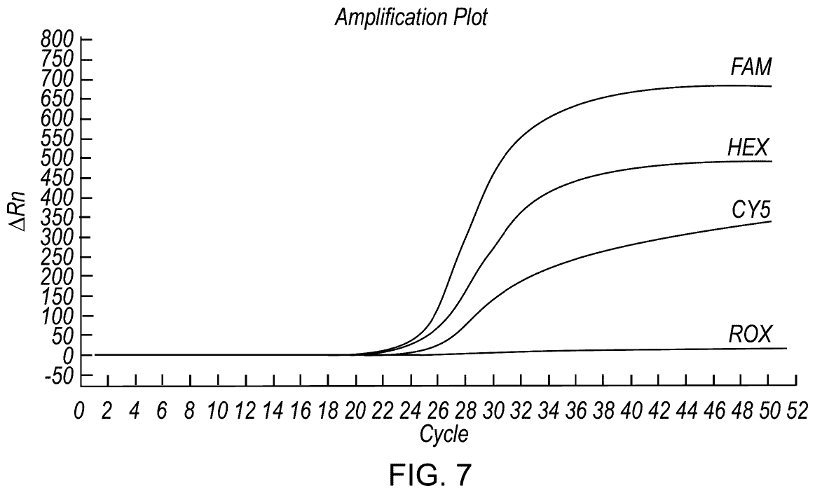

FIG. 7 is a graph showing the change in fluorescence ΔRn versus the cycle number during PCR amplification of a sample containing SARS-CoV-2 Omicron BA.2 variant. The graph shows CY5 fluorescent signal indicating Omicron BA.2 variant is present in the sample. The FAM signal detected RNase P (RP) confirming the sample is a human specimen, and the HEX signal detected Nucleocapsid (NP) in the sample confirming the presence of SARS-CoV-2 in the sample.

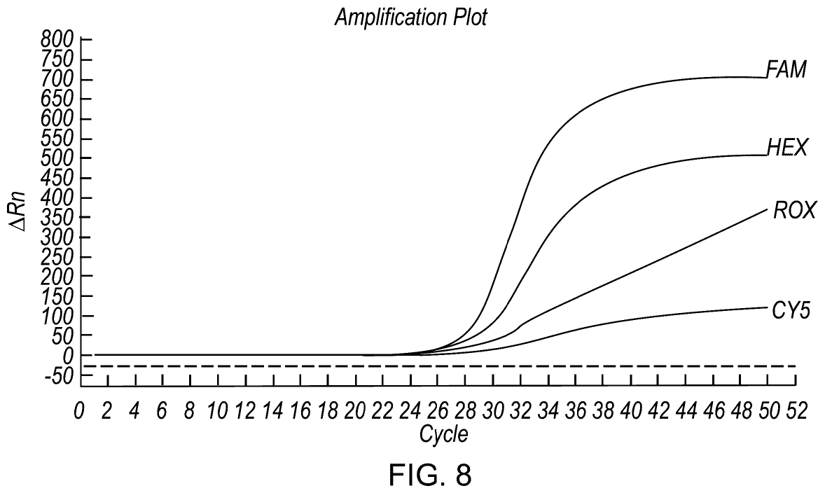

FIG. 8 is a graph showing the change in fluorescence ΔRn versus the cycle number during PCR amplification of a sample containing SARS-CoV-2 Delta variant. The graph shows both ROX and CY5 fluorescent signals indicating the Delta variant is present in the sample. The FAM signal detected RNase P (RP) confirming the sample is a human specimen, and the HEX signal detected Nucleocapsid (NP) in the sample confirming the presence of SARS-CoV-2 in the sample.

The methods provided herein can be tailored to the number of available signal or fluorophore channels of a PCR instrument. In some embodiments, the methods for detecting genomic variants can include three, four, five or six signals for use in PCR machines that have four, five or six signal channels. For example, in one embodiment, a sample can be screened for three different genomic variants using only two signals and two PCR signal channels. In another aspect, a sample can be screened for up to seven different genomic variants using only three signals. The three signals can be three fluorophores such as ROX, CY5 and FAM.

Table 2 below shows the signals for detection of up to seven viral variants using three loci and three fluorophores, ROX, CY5 and FAM in a method of the present invention.

TABLE 2

| Locus/ | Genomic Variant | | | | | | |
|---|---|---|---|---|---|---|---|
| Fluorophore | Variant 1 | Variant 2 | Variant 3 | Variant 4 | Variant 5 | Variant 6 | Variant 7 |
| A/ROX | ROX | No | ROX | ROX | No | ROX | No |
| B/CY5 | No | CY5 | CY5 | CY5 | No | No | CY5 |
| C/FAM | No | No | No | FAM | FAM | FAM | FAM |

Table 3 below shows the signals for detection of eight genomic variants using three loci and three fluorophores wherein one variant is detected when all signals are negative for the three loci being tested. A negative result for all three loci can indicate another SARS-CoV-2 variant is in the sample, but such variant does not include Locus A, Locus B, or Locus C. This embodiment is particularly useful if a sample is extracted from a human or other vertebrate known to have tested positive for SARS-CoV-2 in a prior test and there is a desire to identify the specific SARS-CoV-2 variant. In another embodiment, one of the three loci can be a control locus to confirm that the sample is from a human or other vertebrate, (e.g., an RP locus) or that it is a SARS-CoV-2 virus (e.g., an NP locus present in all SARS-CoV-2 variants).

TABLE 3

| Locus/ | Genomic Variant | | | | | | | |
|---|---|---|---|---|---|---|---|---|
| Fluorophore | V1 | V2 | V3 | V4 | V5 | V6 | V7 | V8 |
| A/ROX | ROX | No | ROX | ROX | No | ROX | No | No |
| B/CY5 | No | CY5 | CY5 | CY5 | No | No | CY5 | No |
| C/FAM | No | No | No | FAM | FAM | FAM | FAM | No |

In other embodiments, provided herein are methods for diagnosing and treating a patient having a SARS-Cov-2 infection. In one aspect a method for diagnosing and treating a patient having a SARS-CoV-2 infection comprises (a) obtaining a sample from the patient; (b) adding the sample and primers to a multiplex PCR machine wherein each primer is capable of extending and duplicating a SARS-Cov-2 variant locus; (c) adding a first probe to the multiplex PCR machine wherein the first probe is labeled with a first signal capable of binding to a first SARS-Cov-2 locus; (d) adding a second probe to the multiplex PCR machine wherein the second probe is labeled with a second signal capable of binding to a second SARS-Cov-2 locus but is not capable of binding to the first SARS-Cov-2 locus; (e) adding a third probe to the multiplex PCR machine wherein the third probe is labeled with the first signal wherein the third probe is capable of binding to a third SARS-Cov-2 locus but is not capable of binding to the first or second SARS-Cov-2 loci; (f) adding a fourth probe to the multiplex PCR machine wherein the fourth probe is labeled with the second signal wherein the fourth probe is capable of binding to a fourth SARS-Cov-2 locus present in both of the second and third SARS-Cov-2 variants but not present in the first SARS-Cov-2 variant; (g) performing PCR in the multiplex PCR machine; and (h) detecting the presence of only the first signal, only the second signal, or both the first and second signals wherein detection of only the first signal identifies the presence of the first SARS-Cov-2 variant in the sample, the detection of only the second signal identifies the presence of the second SARS-Cov-2 variant in the sample, and the detection of both the first and second signals identifies the presence of the third SARS-Cov-2 variant in the sample, and (1) if the first SARS-CoV-2 variant is detected, treating the patient with a therapy effective for treating the first SARS-CoV-2 variant; (2) if the second SARS-CoV-2 variant is detected, treating the patient with a therapy effective for treating the second SARS-CoV-2 variant; and (3) if the third SARS-CoV-2 variant is detected, treating the patient with a therapy effective for treating the third SARS-CoV-2 variant.

In one aspect, the therapy for treating the SARS-CoV-2 variant comprises a biologic such as an antibody or monoclonal antibody. In another aspect, the therapy for treating the SARS-CoV-2 variant comprises a small molecule. In still another aspect, a therapy for treating a SARS-CoV-2 variant comprises a combination of monoclonal antibodies, a combination of small molecules, or a combination of one or more monoclonal antibodies and one or more small molecules. In some aspects, the patient having a SARS-CoV-2 Omicron or Delta infection is treated with tixagevimab, cilgavimab, or bebtelovimab, or a combination thereof. In another aspect, the patient having a SARS-CoV-2 Omicron or Delta infection is treated with a composition of one or more small molecules such as, for example, remdesivir or molnupiravir. Convalescent plasma containing anti-SARS-CoV-2 antibodies from one or more patients infected with SAR-CoV-2 can also be used for treating a COVID-19 infection caused by a particular SARS-CoV-2 variant.

In another embodiment, a multiplex dipstick assay can be used to detect genomic variants in accordance with the methods herein. For example, DNA or RNA amplicons of a PCR assay can be attached to a dipstick membrane for rapid detection of genomic variants. The DNA or RNA amplicons can be attached to the dipstick membrane using nucleic acid hybridization and detected with labeled probes. See for example, Dineva et al., J. Clin. Microbiology, Vol. 43, No. 8, 4015-4021 (2005) and Koczula K M, Gallotta A. Lateral flow assays. Essays Biochem. 2016; 60(1):111-120. doi: 10.1042/EBC20150012. In one aspect, four polynucleotides each encoding a genomic locus present in a genomic variant are amplified in a PCR assay. The amplification products are detected using a lateral flow dipstick (LFD) assay. In another aspect, the presence of a genomic variant is detected by interrogating the four loci wherein two different loci are detected through the same signal, i.e., a semi-specific signal, and two loci are detected with two separate specific signals. For each genomic variant to be detected at least one uniquely identifying locus is examined.

In one embodiment, a lateral flow assay may comprise multiple different detectable labels to facilitate the detection of loci or proteins that are generated after transcription and translation of the loci in genomic variants. Examples for such different labels are fluorophores, i.e. as described above, or dyes or labels that are detectable without fluorescence excitation, such as colloidal gold/gold nanoparticles, silver-enhanced colloidal gold particles. See A. B. Nurul Najian et al., Development of multiplex loop mediated isothermal amplification (m-LAMP) label-based gold nanoparticles lateral flow dipstick biosensor for detection of pathogenic Leptospira. Analytica Chimica Acta, Volume 903,

2016, Pages 142-148, and, Jiang et al. J. Dairy Sci. Vol. 103 No. 5 4002-4012 (2020).

Other suitable labels for use in a lateral flow assay include Fluorescein isothiocyanate (FITC), Texas Red, phycoerythrin (PE), PE-Texas Red, Alexa Fluor 488, Carboxyfluorescein Diacetate (CFSE), Propidium iodide (PI), PE-Cy5, PerCP proteins and PerCP conjugates, PerCP-Cy5.5, PE-Cy7, Allophycocyanin (APC), Alexa Fluor 647, and tandem dyes such as APC-Cy5.5 and APC-Cy7.

Conjugation of a label and a probe or antigen that specifically binds to a locus or a protein that is generated after transcription and translation of the locus present in a genomic variant that is to be detected can be accomplished by a variety of linkers, haptens and binding partners and cognate ligands that allow specific labeling of a probe and a targeting agent. Examples include antibodies that bind specifically to FITC, digoxigenin, or biotin. Streptavidin can be used as a binding partner for biotin. Digoxigenin (DIG) can also be used in the methods herein to generate non-radioactive probes, and is an alternative to biotinylation. Like biotin, DIG-labeled probes support both chromogenic and chemiluminescence detection formats. See “Digoxigenin” (2021) Wikipedia.

In another embodiment, different labels or signals can be substituted for different positions on a lateral flow or dipstick assay. The schematic described above for the use of multiple fluorophores to unambiguously detect specific genomic variants through the presence of multiple loci distinctly identifying such variants can be substituted with the detection of specific reaction products or amplification products that result from the presence of such loci at specific positions on a lateral flow assay. A combination of different positions on a lateral flow test can distinctly identify a specific genomic variant that contains the exact combination of such loci.

For example, commercially available primers and InnoLiPA test strips (Innogenetics/Ghent, Belgium) can be used to amplify exons 1-3 of the HLA-B locus after separating diploid genomic DNA samples into their two haploid components. The LiPA-strips (Line-Probe Assay) are simple, linear DNA ‘arrays’ on paper used in lateral-flow-based HLA tissue typing. The two stripes/lines on the left of each strip are test identification and positive control lines and not included in the description below.

Whole Genome Amplification (WGA) is an isothermal strand-displacement amplification based on Phi29 polymerase and random hexamer primers. WGA was carried out on 1 μl of haploextracted DNA according to the manufacturer's protocol (GenomiPhi DNA amplification kit, Amersham Biosciences, Piscataway, NJ). 1 μl of the WGA-amplified DNA was then used for the LiPA PCR.

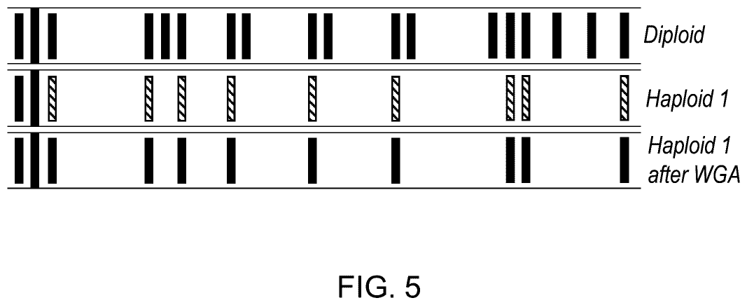

FIG. 5 shows a comparison of LiPA reaction patterns of 16 separate HLA-C loci for diploid DNA identified as Cw05/08 (top), haploid DNA after haplotype-specific extraction (center) and haploid DNA identified as the Cw*0504 allele after haplotype-specific extraction, and haploid DNA identified as the Cw*0504 allele after haplotype-specific extraction and whole genome amplification (bottom). The sample was targeted by HSE for HLA-B*44 but amplified for HLA-C (a 90 kb distance). The distinct haploid reaction pattern consisting of 9 distinct lines representing 9 distinct loci seen in both the center and bottom parts of FIG. 5 uniquely and distinctly identify one of the two haploid genomic variants that are present in the diploid sample as the Cw*0504 allele. If the un-separated, diploid genomic sample is amplified and applied for detection to the lateral flow assay dipstick, the pattern of 16 lines identifies the sample as Cw05/08 (a diploid combination). The observed patterns and HLA-B/HLA-C molecular linkage shown here were confirmed as correct by a family study.

In another embodiment, provided herein are kits for detecting genomic variants in a multiplex assay. In one aspect, the kit comprises polynucleotide primers capable of amplifying a polynucleotide sequence of interest, and polynucleotide probes wherein a first polynucleotide probe is labeled with a first fluorophore and said first probe is only capable of binding to a polynucleotide sequence comprising a first genomic locus, a second polynucleotide probe is labeled with a second fluorophore and said second probe is only capable of binding to a polynucleotide sequence comprising a second genomic locus, a third polynucleotide probe is labeled with the first fluorophore and said third probe is only capable of binding to a polynucleotide sequence comprising a third genomic locus, and a fourth polynucleotide probe is labeled with the second signal and said fourth probe is only capable of binding to a polynucleotide sequence comprising a fourth genomic locus.

In some embodiments, a kit for detecting a SARS-Cov-2 variant in a multiplex polymerase chain reaction may be provided. The kit may include two to eight polynucleotide primers capable of binding to and amplifying one to four polynucleotide sequences comprising at least a portion of a SARS-CoV-2. The kit may include four polynucleotide probes. A first polynucleotide probe may be labeled with a first fluorophore and may only be capable of binding to a first SARS CoV-2 locus. A second polynucleotide probe may be labeled with a second fluorophore and may only be capable of binding to a second SARS-Cov-2 locus. A third polynucleotide probe may be labeled with the first fluorophore and may only be capable of binding to a third SARS-CoV-2 locus. A fourth polynucleotide probe may be labeled with the second fluorophore and may only be capable of binding to a fourth SARS-CoV-2 locus.

In one embodiment, provided herein is a kit for detecting a SARS-Cov-2 variant in a multiplex polymerase chain reaction comprising three sets of polynucleotide primers wherein each set of polynucleotide primers comprises a forward primer and reverse primer capable of amplifying at least a portion of a SARS-CoV-2 variant; and four polynucleotide probes wherein a first polynucleotide probe is labeled with a first fluorophore and said first probe is only capable of binding to a first SARS CoV-2 locus, a second polynucleotide probe is labeled with a second fluorophore and said second probe is only capable of binding to a second SARS-Cov-2 locus, a third polynucleotide probe is labeled with the first fluorophore and said third probe is only capable of binding to a third SARS-CoV-2 locus, and a fourth polynucleotide probe is labeled with the second fluorophore and said fourth probe is only capable of binding to a fourth SARS-CoV-2 locus. The first and second fluorophore should be different fluorophores. In some embodiments, the first and second fluorophore may independently be FAM, HEX, ROX, CY5, NED, VIC, PET and/or LIZ. In some embodiments, the four probes may each, independently, include a quencher. In some embodiments, all four probes may include a quencher. In some embodiments, the quencher(s) for each probe may independently be BMN-Q535, BMN-Q620, black hole quencher, dabcyl, tetramethylrhodamine, or deep dark quencher.

In one aspect, kits are provided comprising a first polynucleotide primer comprising a nucleic acid sequence of SEQ ID NO: 1; a second polynucleotide primer comprising a nucleic acid sequence of SEQ ID NO: 2; a first polynucleotide probe comprising a nucleic acid sequence of SEQ ID NO: 3, a third polynucleotide primer comprising a nucleic acid sequence of SEQ ID NO: 4; a fourth polynucleotide primer comprising a nucleic acid of SEQ ID NO: 5; a second polynucleotide probe comprising a nucleic acid sequence of SEQ ID NO: 6; a fifth polynucleotide primer comprising a nucleic acid sequence of SEQ ID NO: 7; a sixth polynucleotide primer comprising a nucleic acid of SEQ ID NO: 8; a third polynucleotide probe comprising a nucleic acid sequence of SEQ ID NO: 9, and a fourth polynucleotide probe comprising a nucleic acid sequence of SEQ ID NO: 10.

In some embodiments, the kits of the present invention comprise buffer mix, enzyme (or master) mix, primers, probes, a diluent such as nuclease-free water, and positive control polynucleotide sequences.

In one aspect, the components of the kits are frozen, shipped, and stored between-25° C. and −15° C. In one aspect, the buffer mix comprises buffer and deoxynucleotide triphosphates (dNTPs), and the enzyme mix comprises reverse transcriptase and heat-stable polymerase.

The kits provided herein are used to perform qRT-PCR comprising (a) reverse transcription of target RNA, such as SARS-CoV-2 RNA and human RNase P (control), to cDNA, (b) PCR amplification of target cDNA, quantitative detection of PCR amplification products by fluorescent dye labeled probes, such as, for example, FAM, HEX, ROX and CY5.

### EXAMPLES

The following illustrative examples are representative of embodiments of compositions and methods described herein and are not meant to be limiting in any way.

### Example 1

Reagents were stored between −25° C. and −15° C., and samples were thawed by pipetting or gentle vortexing and centrifuged briefly before use. A total reaction volume of 20 μl for each sample (or control) tested included 10 μl of buffer mix (BM), 0.2 μl of enzyme mix (EM), primer and probe mix (PPM), sample (or control) 7.5 μl, and 0.3 μl of nuclease-free water. The real-time PCR settings were reaction volume 20 μl, default ramp rate setting, and no passive resistance. The probe targeting SARS-CoV-2 gene N1 (control) was labeled with fluorophore reporter FAM, and at the 3′ end of the probe quencher BMN-Q535; the probe targeting human RNase P was labeled with fluorophore reporter HEX, and at the 3′ end of the probe quencher BMN-Q535; the probe targeting SARS-CoV-2 Omicron BA.1 specific deletion 143 (D143) was labeled with fluorophore reporter ROX, and at the 3′ end of the probe quencher BMN-Q620; and the probe targeting SARS-CoV-2 Delta specific deletion D157 was also labeled with fluorophore reporter ROX, and at the 3′ end of the probe quencher BMN-Q620. A third probe targeting Omicron BA.2 specific deletion D24 was also labeled with fluorophore reporter CY5, and at the 3′ end of the probe quencher BMN-Q620, and a fourth probe (drop-off probe for BA.1) targeting wild type 143 locus (wt143) was labeled with fluorophore reporter CY5, and at the 3′ end of the probe the quencher BMN-Q620.

The reactions were incubated as shown in the following Table 4.

TABLE 4

| Step | Stage | Cycles | Acquisition | Temp | Time | |
|---|---|---|---|---|---|---|
| RT | Hold | 1 | — | 50° C. | 10 | min |
| Denaturation | Hold | 1 | — | 95° C. | 2 | min |
| Denaturation | Cycling | 50 | — | 95° C. | 5 | sec |
| Amplification | | | YES | 60° C. | 30 | sec |

The threshold of the qPCR instrument was adjusted to 10% of the signal intensity of the maximum value of the positive control. Samples reaching less than 10% of the signal intensity of the positive controls were regarded as negative. Results are provided in Table 5 below.

TABLE 5

| | CoV-2 | Human | CoV-2 | CoV-2 | |
|---|---|---|---|---|---|
| | N1 | RNase P | Omicron | Delta | |
| | FAM | HEX | ROX | CY5 | |
| Sample | Probe | Probe | Probe | Probe | Result |
| 1 | POS | POS | POS | NEG | SARS-CoV-2 |
| | | or NEG | | | Omicron BA.1 |
| | | | | | Variant |
| | | | | | Detected |
| 2 | POS | POS | NEG | POS | SARS-CoV-2 |
| | | or NEG | | | Omicron BA.2 |
| | | | | | Variant |
| | | | | | Detected |
| 3 | POS | POS | NEG | NEG | No Omicron |
| | | or NEG | | | or Delta |
| | | | | | Variant |
| | | | | | Detected |
| 4 | POS | POS | POS | POS | SARS-CoV-2 |
| | | | | | Delta |
| | | | | | Variant |
| | | | | | Detected |
| 5 | NEG | POS | NEG | NEG | SARS-CoV-2 |
| | | | | | Not Detected |
| 6 | NEG | NEG | NEG | NEG | Invalid |
| | | | | | Repeat Test |

All of the publications and patent applications and patents cited in this specification are herein incorporated by reference in their entirety.

Certain specific details are set forth herein in order to provide a thorough understanding of various embodiments of the present invention. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments. While several particular forms of the disclosure have been illustrated and described, it will be apparent that various modifications and combinations of the disclosure detailed in the text and drawings can be made without departing from the spirit and scope of the disclosure.

What is claimed is:

1. A method for detecting a SARS-CoV-2 variant in a sample comprising:
(a) adding three primer sets to a multiplex PCR machine wherein each primer set is capable of extending and duplicating a polynucleotide sequence comprising a SARS-Cov-2 variant locus; (b) adding a first probe to the multiplex PCR machine wherein the first probe is labeled with a first signal capable of binding to a first SARS-Cov-2 locus;
(c) adding a second probe to the multiplex PCR machine wherein the second probe is labeled with a second signal capable of binding to a second SARS-Cov-2 locus but is not capable of binding to the first SARS-Cov-2 locus;
(d) adding a third probe to the multiplex PCR machine wherein the third probe is labeled with the first signal wherein the third probe is capable of binding to a third SARS-Cov-2 locus but is not capable of binding to the first or second SARS-Cov-2 loci;
(e) adding a fourth probe to the multiplex PCR machine wherein the fourth probe is labeled with the second signal wherein the fourth probe is capable of binding to a fourth SARS-Cov-2 locus present in both of the second and third SARS-Cov-2 variants but not present in the first SARS-Cov-2 variant;
(f) performing PCR in the multiplex PCR machine; and
(g) detecting the presence of only the first signal, only the second signal, or both the first and second signals wherein detection of only the first signal identifies the presence of the first SARS-Cov-2 variant in the sample, the detection of only the second signal identifies the presence of the second SARS-Cov-2 variant in the sample, and the detection of both the first and second signals identifies the presence of the third SARS-Cov-2 variant in the sample.

2. The method of claim 1, wherein the first and second signals are fluorophores.

3. The method of claim 2, wherein the fluorophores are selected from the group consisting of FAM, HEX, ROX, CY5, NED, VIC, PET and LIZ.

4. The method of claim 2, wherein the first signal is ROX and the second signal is CY5.

5. The method of claim 1, wherein the SARS-Cov-2 locus is selected from the group consisting of an Omicron variant, a Delta variant, and combinations thereof.

6. The method of claim 1, wherein the SARS-Cov-2 variant is selected from the group consisting of Omicron BA.1, Omicron BA1.1, Omicron BA.2 and Delta.

7. The method of claim 1, wherein the first SARS-Cov-2 locus is the D143 locus in the Omicron BA.1 variant.

8. The method of claim 1, wherein the second SARS-Cov-2 locus is D24 in the Omicron BA.2 variant.

9. The method of claim 1, wherein the third SARS-Cov-2 locus is D157 in the Delta variant.

10. The method of claim 1, wherein the fourth SARS-Cov-2 locus is WT143 present in the Omicron BA.2 variant and the Delta variant.

11. The method of claim 1, wherein the sample is a human sample.

12. A method for treating a patient for SARS-Cov-2 comprising
(a) obtaining a sample from the patient;
(b) adding the sample and primers to a multiplex PCR machine wherein each primer is capable of extending and duplicating a SARS-Cov-2 variant locus;
(c) adding a first probe to the multiplex PCR machine wherein the first probe is labeled with a first signal capable of binding to a first SARS-Cov-2 locus;
(d) adding a second probe to the multiplex PCR machine wherein the second probe is labeled with a second signal capable of binding to a second SARS-Cov-2 locus but is not capable of binding to the first SARS-Cov-2 locus;
(e) adding a third probe to the multiplex PCR machine wherein the third probe is labeled with the first signal wherein the third probe is capable of binding to a third SARS-Cov-2 locus but is not capable of binding to the first or second SARS-Cov-2 loci;
(f) adding a fourth probe to the multiplex PCR machine wherein the fourth probe is labeled with the second signal wherein the fourth probe is capable of binding to a fourth SARS-Cov-2 locus present in both of the second and third SARS-Cov-2 variants but not present in the first SARS-Cov-2 variant;
(g) performing PCR in the multiplex PCR machine; and
(h) detecting the presence of only the first signal, only the second signal, or both the first and second signals wherein detection of only the first signal identifies the presence of the first SARS-Cov-2 variant in the sample, the detection of only the second signal identifies the presence of the second SARS-Cov-2 variant in the sample, and the detection of both the first and second signals identifies the presence of the third SARS-Cov-2 variant in the sample, and
(1) if the first SARS-CoV-2 variant is detected, treating the patient with a therapy effective against the first SARS-CoV-2 variant;
(2) if the second SARS-CoV-2 variant is detected, treating the patient with a therapy effective against the second SARS-CoV-2 variant; and
(3) if the third SARS-CoV-2 variant is detected, treating the patient with a therapy effective against the third SARS-CoV-2 variant.

13. The method of claim 12, wherein the therapy effective against the first SARS-CoV-2 variant, second SARS-CoV-2 variant, and/or third SARS-CoV-2 variant is a monoclonal antibody, a small molecule, or a combination thereof.

14. The method of claim 12, wherein the first SARS-CoV-2 variant is Omicron BA.1.

15. The method of claim 12, wherein the second SARS-CoV-2 variant is Omicron BA.2

16. The method of claim 12, wherein the third SARS-CoV-2 variant is Delta.

17. A kit for detecting a SARS-Cov-2 variant in a multiplex polymerase chain reaction comprising:
two to eight polynucleotide primers capable of binding to and amplifying one to four polynucleotide sequences comprising at least a portion of a SARS-CoV-2; and
four polynucleotide probes wherein a first polynucleotide probe is labeled with a first fluorophore and said first polynucleotide probe is only capable of binding to a first SARS CoV-2 locus, a second polynucleotide probe is labeled with a second fluorophore and said second polynucleotide probe is only capable of binding to a second SARS-Cov-2 locus, a third polynucleotide probe is labeled with the first fluorophore and said third polynucleotide probe is only capable of binding to a third SARS-CoV-2 locus, and a fourth polynucleotide probe is labeled with the second signal and said fourth polynucleotide probe is only capable of binding to a fourth SARS-CoV-2 locus.

18. The kit of claim 17, wherein the first, second, third and fourth polynucleotide probes are labeled with a fluorophore selected from the group consisting of FAM, HEX, ROX, CY5, NED, VIC, PET and LIZ.

19. The kit of claim 17, wherein the first, second, third and fourth polynucleotide probes include a quencher selected from the group consisting of BMN-Q535, BMN-Q620, black hole quencher, dabcyl, tetramethylrhodamine and deep dark quencher.

20. The kit of claim 17, wherein:
(a) the first set of polynucleotide primers comprises a nucleic acid sequence of SEQ ID NO: 1 and a nucleic acid sequence of SEQ ID NO:2;
(b) the second set of polynucleotide primers comprises a nucleic acid sequence of SEQ ID NO: 4 and a nucleic acid sequence of SEQ ID NO:5;
(c) a third set of primers comprises a nucleic acid sequence of SEQ ID NO: 7 and a nucleic acid sequence of SEQ ID NO: 8;
(d) the first probe comprises a nucleic acid sequence of SEQ ID NO: 3;
(e) the second probe comprises a nucleic acid sequence of SEQ ID NO: 6;
(f) the third probe comprises a nucleic acid sequence of SEQ ID NO: 9; and
(g) a fourth probe comprises a nucleic acid sequence of SEQ ID NO: 10.
